(19) United States
(12) Patent Application Publication
(10) Pub. No.: US 2018/0221529 A1
(43) Pub. Date: Aug. 9, 2018

(54) SWEET ATMOSPHERE HUMIDIFIER

(71) Applicant: Pao-Tien Chiu, New Taipei (TW)

(72) Inventor: Pao-Tien Chiu, New Taipei (TW)

(21) Appl. No.: 15/428,526

(22) Filed: Feb. 9, 2017

(51) Int. Cl. A61L 9/14; F24F 6/12

(52) U.S. Cl. CPC A61L 9/14; F24F 2006/006; A61L 2209/134; F24F 6/12

(57) ABSTRACT

A sweet atmosphere humidifier comprises a base, a control module, an air-guiding cap, a water box and a decorative cover. The air-guiding cap is provided with a chamber and a first conductive terminal exposed inside the chamber. The water box is provided with a liquid storage tank, an atomizing unit inside the liquid storage tank, and a second conductive terminal on the external surface of the water box. The base, the control module, the air-guiding cap and the water box are mounted inside the decorative cover. In the sweet atmosphere humidifier, the atomizing unit is possibly provided in the water box to be replaced conveniently by fitting the first conductive terminal to the second conductive terminal. Moreover, the appearance profile of the decorative cover may be further fitted to the shape of base, control module, air-guiding cap and water box, so as to obtain more interesting appearance.

### FIELD OF THE INVENTION

The present invention is related to a sweet atmosphere humidifier, particularly to a sweet atmosphere humidifier being convenient for making up liquid, such as water and essential oil, while interesting in appearance.

### BACKGROUND OF THE INVENTION

It is found that the problem of insufficient humidity appears in some areas where air is dry or in a room where air conditioner is turned on. Nowadays, many industries have researched and developed indoor humidifiers. The humidifiers may be used by people to spray atomized water vapor indoors, so as to increase humidity. Thereby, indisposition due to extremely dry air may be avoided. In consideration of the process of use of humidifiers, it is allowed for the user to add essential oil into the humidifier, such that the essential oil is emanated to the indoor environment together with the atomized water vapor. Thus, fatigue of the user may be relieved via sweetness emanated from the essential oil. Moreover, there is a need for making up water and essential oil after the humidifier is used for a period of time. Therefore, the structure of a general household small-sized humidifier, for the most part, includes a foundation, a liquid storage tank and a housing. The liquid storage tank is encased between the foundation and the housing, such that the user is allowed to pour water and essential oil into the liquid storage tank after the housing is dismantled.

Further, according to the requirement for the appearance of product in the market, there is a humidifier to be fitted, in the exterior thereof, with a specially-shaped housing currently, such that an orifice for atomized vapor of the humidifier is capable of being combined with an exhaust port of the specially-shaped housing, so as to form an appearance of a meaningful pattern (for example, dinosaur or volcano). Thereby, purchase intention of the user is enhanced. Furthermore, the volume and material of the humidifier may be also designed by the industry currently in accordance with the style of interior decoration, in such a way that the humidifier is adapted to a larger indoor space by increasing in volume (for example, a hotel), and is adapted to decorative material of indoor space by using various materials (for example, metal). For instance, U.S. publication patent no. 20160243576A1 disclosed a system including a diffuser and a cover. The diffuser is provided with a diffuser chamber, an outlet, a connecting port and two first mounting elements. Water and essential oil may be stored inside the diffuser. The outlet is communicated with the diffuser chamber. The connecting port may be connected to a power supply device so as to supply the diffuser with operating power. The two first mounting elements are provided at two opposite exteriors of the diffuser chamber, respectively. On the other hand, the cover is provided with a chamber, two second mounting elements, a plurality of detachable decorative features and an opening. The chamber is provided at the bottom of the cover. The two mounting elements are provided at opposite locations within the chamber. The detachable features are provided on the exterior of the cover. The opening is provided on the exterior of the cover and communicated with the interior of the chamber. In the practical use of the system, the diffuser is placed into the chamber from the bottom of the cover. The fixed relationship (such as a button, rod, knob, turntable, and any structure for mounting and fixing) of the diffuser and the cover is formed by fitting the two first mounting elements to the two second mounting elements. Thereby, in the operation of the diffuser, atomized water and essential oil may be diffused to the indoor environment via the outlet and the opening.

It is known, however, from the above technical content of US20160243576A1, that it is necessary to place the diffuser into the chamber from the bottom of the cover when the system is used. After water within the diffuser is exhausted completely, it is necessary for the user to lift the whole cover, so as to draw out the diffuser and then refill water and essential oil into the diffuser. In this connection, the volume of the diffuser and the cover is larger than that of a general household small-sized humidifier, as well as metal, ceramics, wood, stone and so on may be adopted as the material for manufacturing the cover so as to be suitable for the interior decoration. In view of this, it is not only dangerous to the user in making up water and essential oil, but also unduly difficult in making up water and essential oil for the diffuser, due to lifting the whole cover. Moreover, accelerated wear or damage to the cover or the diffuser may be possible after repeatedly making up the water and essential oil.

Therefore, how to design a sweet atmosphere humidifier capable of facilitating the user to make up water and essential oil rapidly is obviously a problem to be solved.

### SUMMARY OF THE INVENTION

It is the main object of the present invention to solve the problem of difficulty in making up water and essential oil in the conventional structure.

For achieving the above object, the present invention provides a sweet atmosphere humidifier, comprising a base, a control module, an air-guiding cap, a water box and a decorative cover. The control module is provided on the base. The air-guiding cap is provided on the base, and the air-guiding cap is provided with a chamber formed within the air-guiding cap, an installation mouth communicated with the chamber, an exhaust vent communicated with the chamber and provided on the top of the air-guiding cap, and a first conductive terminal exposed inside the chamber and electrically connected to the control module. The water box is selectively placed into the chamber through the installation mouth. The water box is provided with a liquid storage tank, an atomizing unit provided inside the liquid storage tank, and a second conductive terminal provided on the external surface of the water box while contacted with the first conductive terminal after the water box is placed into the chamber such that electric power may be obtained by the atomizing unit. The decorative cover is mounted to the base so as to cover the control module, the air-guiding cap and the water box. The decorative cover is openly provided at the side thereof with a gap allowing the water box to pass there through and to be placed into or drawn out of the chamber.

In one embodiment, the water box is provided with a water box cover mounted to a part of the liquid storage tank exposed outside of the chamber.

In one embodiment, the water box is provided with a bottle cap adaptor, which is mounted to a part of the liquid storage tank exposed outside of the chamber and is provide for a water bottle to be mounted thereto.

In one embodiment, the liquid storage tank is provided with a first region located inside the chamber, a second region located outside of the chamber, and at least one water delivery pipeline located between the first region and the second region.

In one embodiment, the control module is provided with a switch element, which is electrically connected to the first conductive terminal and is capable of controlling the atomizing unit via the first conductive terminal and the second conductive terminal. Further, the control module is provided with a power connecting port, which is electrically connected to the first conductive terminal and is provided for a power supply device to be electrically connected thereto so as to obtain electric power.

In one embodiment, the base and the air-guiding cap are integrally formed.

In one embodiment, the decorative cover is provided with a side cover, which is mounted to the gap and provided for covering one side of the water box.

In one embodiment, the chamber is provided with an installation channel communicated with the installation mouth and provided for the water box to be placed therein, and an air-guiding space communicated with the installation channel and provided for the exhaust vent to be located at the center of the top thereof

In light of above technical contents, the present invention is provided with features, in comparison with the convention art, as follows.

In the present invention, the water box may be placed into the chamber through the gap at the side of the decorative cover, and the atomizing unit is possibly provided in the water box to be replaced conveniently through fitting the first conductive terminal to the second conductive terminal. Moreover, the appearance profile of the decorative cover may be further manufactured to fit the combined shape of the base, the control module, the air-guiding cap and the water box, so as to obtain more interesting appearance of the present invention.

### BRIEF DESCRIPTION OF THE DRAWINGS

FIG. 1 is a perspective diagram (I) of a part of components of the present invention.

FIG. 2 is a perspective exploded diagram (I) of a part of components of the present invention.

FIG. 3 is a perspective diagram (II) of a part of components of the present invention.

FIG. 4 is a perspective exploded diagram (II) of a part of components of the present invention.

FIG. 5 is a side cross sectional view of the present invention.

FIG. 6 is a top cross sectional view of the present invention.

FIG. 7 is a top cross sectional view of a water box of the present invention.

FIG. 8 is a side cross sectional view taken along a section line 8 in

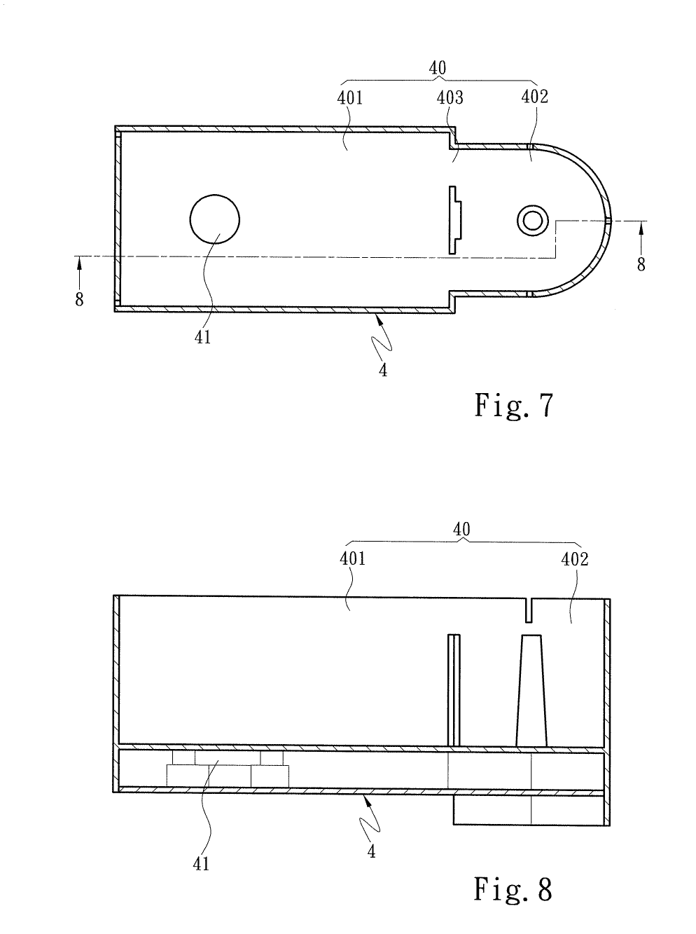

FIG. 7 of the present invention.

FIG. 9 is a side view of the present invention.

FIG. 10 is a perspective diagram of another embodiment of the present invention.

### DETAILED DESCRIPTION OF THE PREFERRED EMBODIMENTS

The detailed description and technical content of the present invention will now be described in combination with drawings as follows.

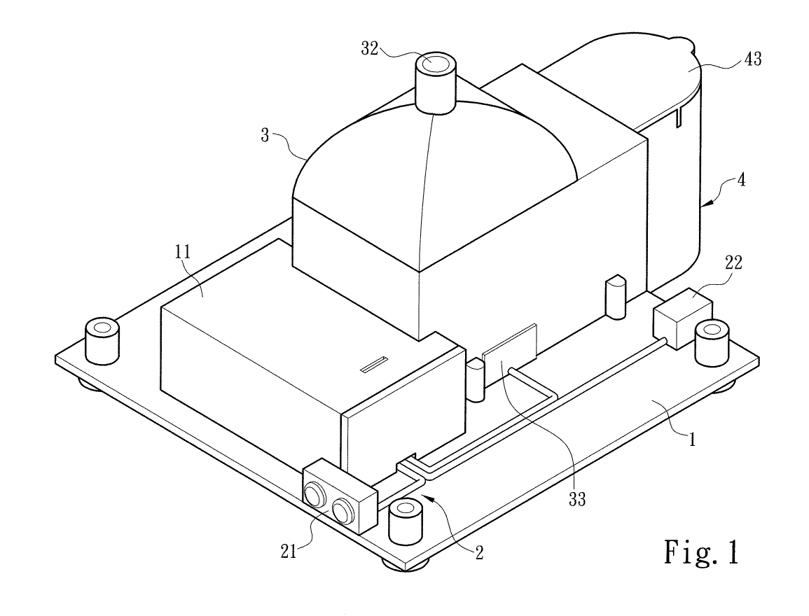

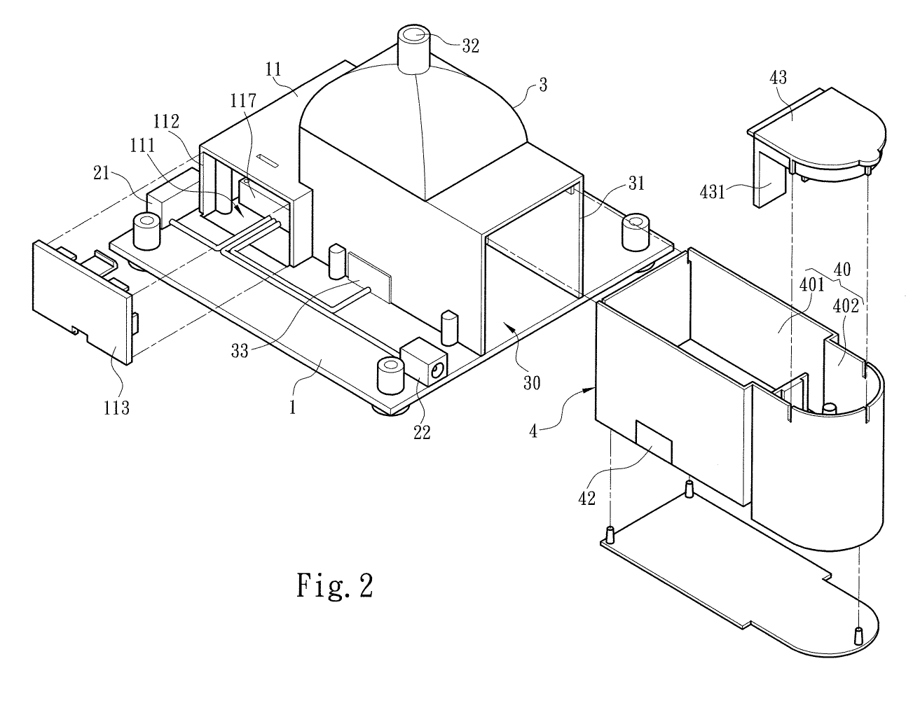

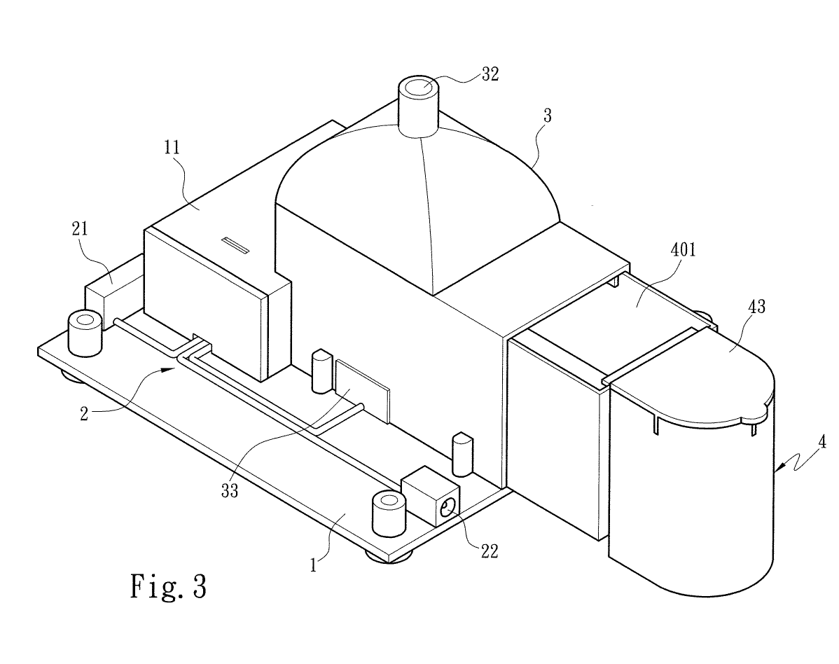

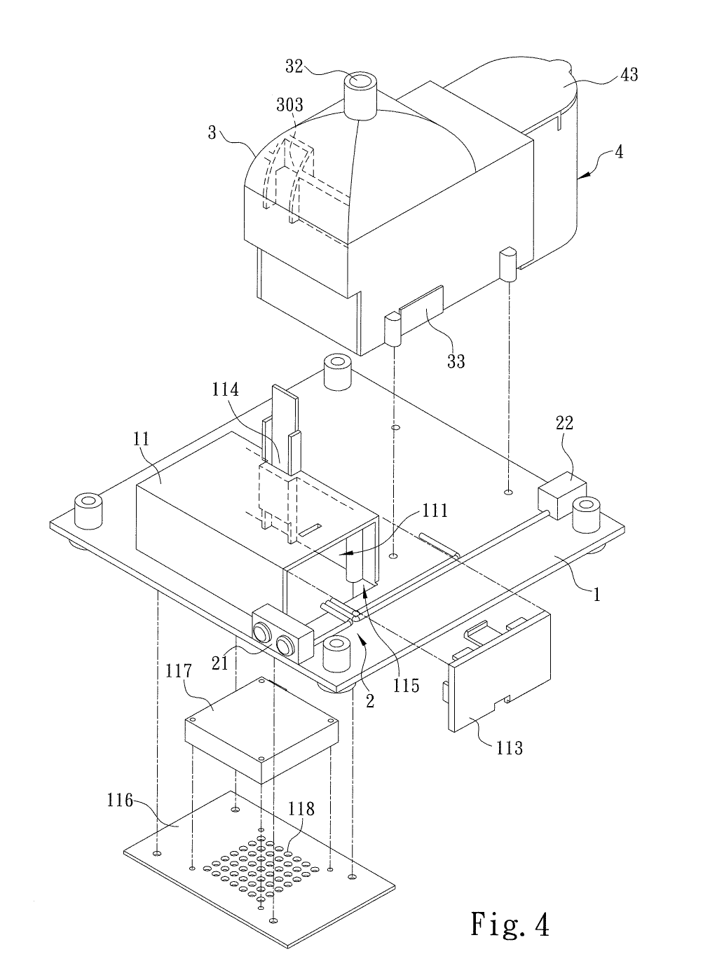

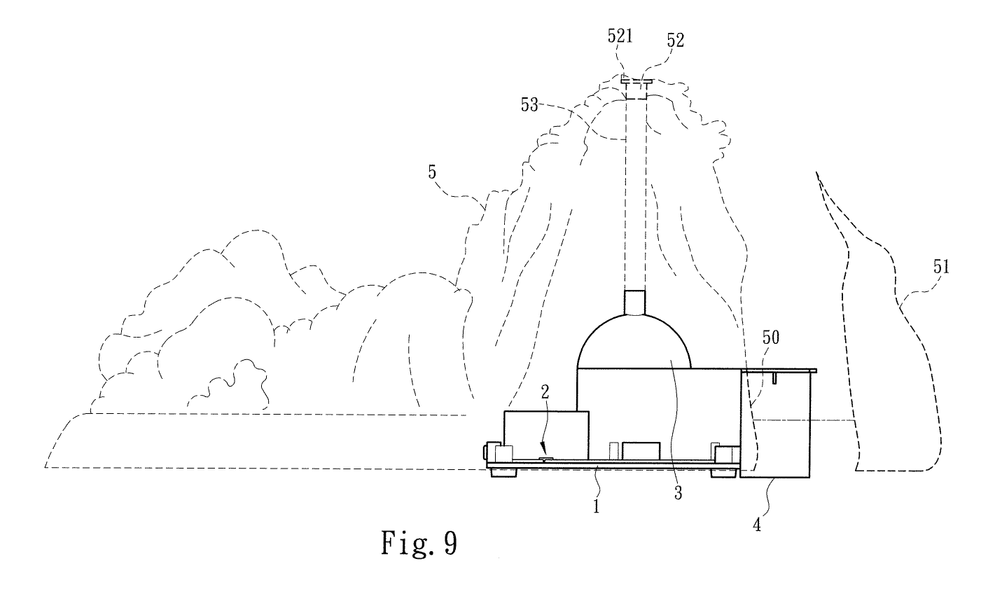

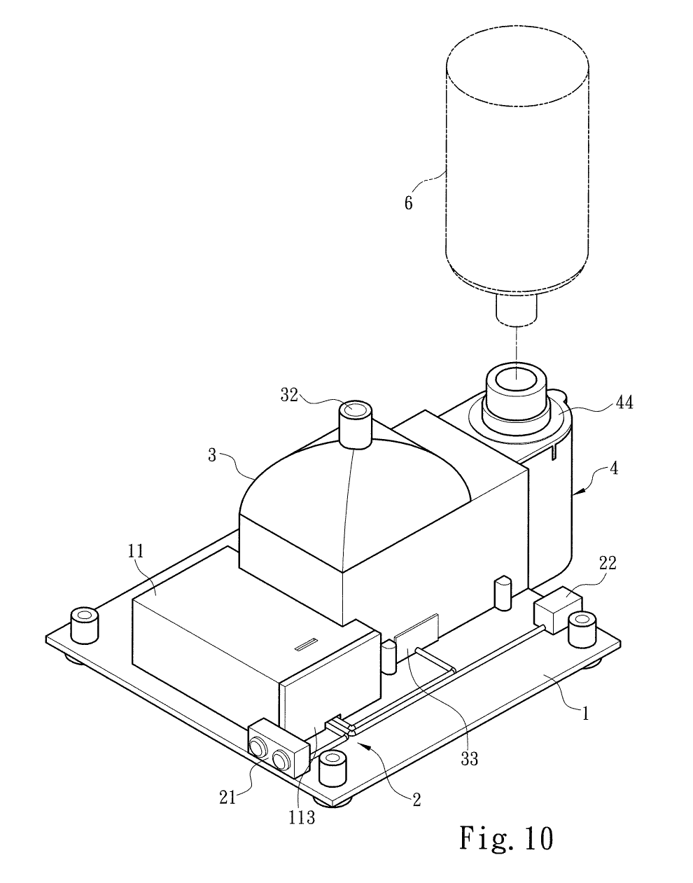

Referring to FIGS. 1 to 9, the present invention provides a sweet atmosphere humidifier, comprising a base 1, a control module 2, an air-guiding cap 3, a water box 4 and a decorative cover 5. The base 1 is presented, in its entirety, in the form of a plate, while the control module 2 is provided on the surface of the base 1. In one embodiment, the control module 2 is provided with a switch element 21, a power connecting port 22 and a microprocessor (not illustrated in the figures). The switch element 21, the power connecting port 22 and the microprocessor are all provided on the surface of the base 1, while the switch element 21 and the power connecting port 22 are both electrically connected to the microprocessor. Specifically, the connecting lines among the switch element 21, the power connecting port 22 and the microprocessor are laid on the surface of the base 1 or on the surface of the decorative cover 5. In one embodiment, the base 1 is provided with an accommodating room 11. As illustrated in FIG. 2, the accommodating room 11 is provided with an installation space 111, an opening 112 and a cover plate 113. The installation space 111 is formed within the accommodating room 11 and provided for the microprocessor to be located therein. The opening 112 is formed at one side of the installation space 111. The cover plate 113 may be mounted to the opening 112. The connecting lines among the switch element 21, the power connecting port 22 and the microprocessor are then allowed to pass through the side of the cover plate 113 (as illustrated in FIG. 3). It should be noted that, however, the connecting lines among the switch element 21, the power connecting port 22 and the microprocessor, illustrated in FIGS. 1 to 4, 6 and 10 may be only one embodiment of the present invention. The connecting lines among the switch element 21, the power connecting port 22 and the microprocessor may be provided in a wired manner or a wireless manner depending upon the requirement of the user. The switch element 21 and the power connecting port 22 may be provided on the surface of the base 1 in any direction, or separated from the surface of the base 1 (for instance, connected to the microprocessor via at least one transmission line while far away from the surface of the base 1).

On the other hand, the air-guiding cap 3 is provided with a chamber 30, an installation mouth 31, an exhaust vent 32 and a first conductive terminal 33. The chamber 30 is formed within the air-guiding cap 3. The installation mouth 31 is provided at the side of the air-guiding cap 3 and communicated with the chamber 30. The exhaust vent 32 is provided on the top of the air-guiding cap 3 and communicated with the chamber 30. The first conductive terminal 33 is exposed inside the chamber 30 and electrically connected to the control module 2. In one embodiment, the air-guiding cap 3 and the accommodating room 11 are integrally formed, while the air-guiding cap 3 and the accommodating room 11 may be mounted or dismounted via the surface of the base 1. In addition, the water box 4 is provided with a liquid storage tank 40, an atomizing unit 41 and a second conductive terminal 42. The liquid storage tank 40 is formed within the water box 4. The atomizing unit 41 is provided inside the liquid storage tank 40. The second conductive terminal 42 is then provided on the external surface of the water box 4, and substantially the same as the first conductive terminal 33 in shape. The atomizing unit 41 is, in terms of constituting structure, composed of an atomizing plate, a silica gel ring and a fixed base. The atomizing unit 41 is possible to scatter liquid into atomized particles by vibration of acoustic wave in an unheated condition, while the switch element 21 may be used for controlling on/off of the atomizing unit 41 and intensity of atomization. Specifically, the chamber 30 is provided with an installation channel 301 communicated with the installation mouth 31, an air-guiding space 302 located above the installation channel 301 while provided for the exhaust vent 32 to be located at the center of the top thereof In this case, the shape of the installation channel 301 is substantially the same as that of the water box 4 (as illustrated in FIG. 2), such that the water box 4 may be placed into the installation channel 301 through the installation mouth 31, and the atomizing unit 41 may be further aligned with the exhaust vent 32 at the center of the air-guiding space 302. In one embodiment, moreover, the chamber 30 is provided with an inserting hole 303 located in the inner wall of the air-guiding space 302. The accommodating room 11 is provided with an air delivery pipeline 114, a mounting mouth 115, a bottom plate 116 and a fan 117. The inserting hole 303 is communicated to the exterior of the air-guiding cap 3 from the air-guiding space 302. The air delivery pipeline 114 is provided at the side of the accommodating room 11, while communicated between the installation space 111 and the inserting hole 303. The mounting mouth 115 is provided at the bottom of the accommodating room 11. The bottom plate 116 is mounted to the mounting mouth 115 and provided with a plurality of ventilation ports 118. The fan 117 is then mounted inside the accommodating room 11 and electrically connected to the control module 2. In this connection, it is possible for the user to dismantle the bottom plate 116 so as to maintain the fan 117. Further, the fan 117 is allowed to introduce external air, through the ventilation ports 118, into the air-guiding space 302 via the installation space 111, the air delivery pipeline 114 and the inserting hole 303, such that the atomized particles produced by the atomizing unit 41 within the air-guiding space 302 may be discharged through the exhaust vent 32.

Finally, referring to FIG. 9 further, the decorative cover 5 is provided with an accommodating space (not illustrated in the figure) and a gap 50. The accommodating space is formed by recessing upward from the bottom of the decorative cover 5. The gap 50 is then provided at the side of the decorative cover 5, while communicated to the interior of the accommodating space. Specifically, the decorative cover 5 may be provided with a fixing part 52 and a conduit 53. The fixing part 52 is formed at inner side thereof with a through-hole 521. The through-hole 521 and the exhaust vent 32 are substantially the same in shape, while the exhaust vent 32 may be aligned with the through-hole 521 after the base 1, the control module 2 and the air-guiding cap 3 have been combined together and placed into the accommodating space. Thus, the conduit 53 may be connected between the exhaust vent 32 and the fixing part 52, such that a communicating relationship is formed between the exhaust vent 32 and the through-hole 521. It should be noted here that, although the decorative cover 5 is drawn by dotted lines in FIG. 9, the dotted lines do not indicate that the decorative cover 5 is not included in the scope of the present invention, but imply that the decorative cover 5 is not limited to the mountain drawn in FIG. 9 instead; that is to say, the decorative cover 5 may be manufactured as a mountain, ship, car, mural, cartoon character, or any decorative appearance design, depending upon the requirement of the manufacturer.

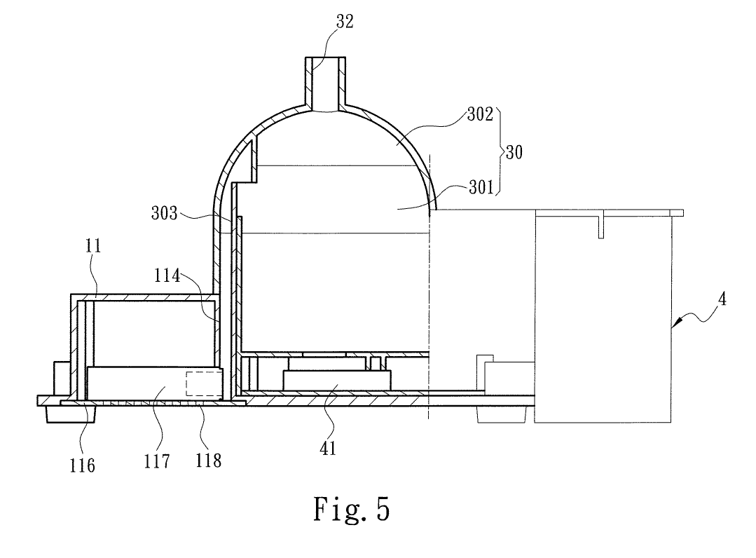

The structure composed of the base 1, the control module 2, the air-guiding cap 3, the water box 4 and the decorative cover 5 is clear from the technical content described above. Here, the process of the use of the sweet atmosphere humidifier and other embodiments occur due to this process of use will be further described for the present invention. In the practical use of the sweet atmosphere humidifier, as illustrated in FIGS. 1 to 4, the sweet atmosphere humidifier may be used for one of two purposes of humidification and sweet atmospheric humidification. In one embodiment, the liquid storage tank 40 is provided with a first region 401, a second region 402 and at least one water delivery pipeline 403. The first region 401 and the second region 402 are connected to each other (as illustrated in FIGS. 5 to 8). The water delivery pipeline 403 is then provided between the first region 401 and the second region 402. When the sweet atmosphere humidifier is used only for humidification, the first region 401 and the second region 402 are both used for storing water; nevertheless, when the sweet atmosphere humidifier is used for sweet atmospheric humidification, the first region 401 is used for storing water and essential oil, while the second region 402 is used for storing water. In this case, the first region 401 may be larger than the second region 402 in volume. Thereby, the storage volume of essential oil may be increased via the first region 401 in the present invention, so as to prolong service time of essential oil in sweet atmospheric humidity, and then prevent the problem of extremely rapid loss of essential oil in a general small-sized diffuser from being presented in the sweet atmosphere humidifier. After liquid (i.e., only water, or both water and essential oil being poured) is poured into the liquid storage tank 40, it is possible for the user to place the water box 4 into the installation channel 301 via the gap 50 and the installation mouth 31, such that the atomizing unit 41 within the liquid storage tank 40 is aligned with the exhaust vent 32 at the top of the air-guiding space 302, and that the second conductive terminal 42 is electrically connected to the first conductive terminal 33. In detail, when the second conductive terminal 42 is electrically connected to the first conductive terminal 33, the atomizing unit 41 is subsequently electrically connected to the switch element 21, the power connecting port 22, the microprocessor and the fan 117. Thus, electric power supplied by a power supply device may be then obtained by the switch element 21, the microprocessor, the atomizing unit 41 and the fan 117 via the power connecting port 22. Conversely, if electrical connection is not formed between the first conductive terminal 33 and the second conductive terminal 42, the switch element 21, the microprocessor and the atomizing unit 41 could not be operated. In other words, whether the first conductive terminal 33 is contacted with the second conductive terminal 42 is essential for the sweet atmosphere humidifier. Moreover, the decorative cover 5 is further provided with a side cover 51, as illustrated in FIG. 9. The side cover 51 may be mounted to the gap 50, so as to cover the exposed portion of the water box 4, such that a whole appearance (such as a mountain, ship, car, mural or cartoon character) is further presented for the decorative cover 5.

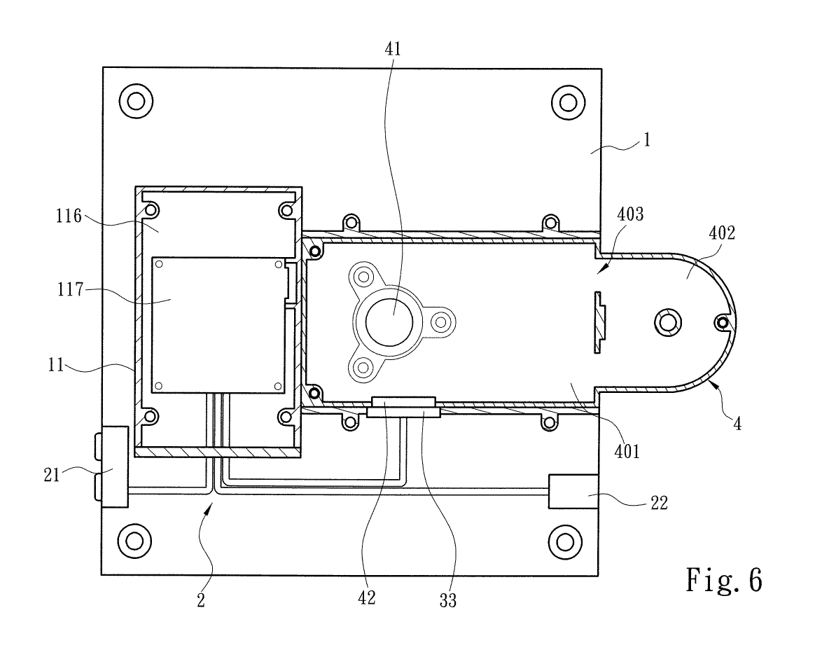

Subsequently, after the water box 4 is placed into the chamber 30 so as to form electrical connection between the first conductive terminal 33 and the second conductive terminal 42 (as illustrated in FIG. 6), it is possible for the user to operate the switch element 21, in such a way that the microprocessor is allowed to control the atomizing unit 41 to atomize liquid and that the fan 117 is allowed to discharge atomized particles, obtained by atomizing liquid, to the exterior of the through-hole 521 through the exhaust vent 32 and the conduit 53 (as illustrated in FIG. 9). If water within the liquid storage tank 40 is insufficient, it is possible for the user to draw the water box 4 out of the installation channel 301 partially (i.e., the water box 4 being drawn out of the installation channel 301 incompletely), and then pour water into the liquid storage tank 40. When the water box 4 is placed into the chamber 30, the first region 401 is located inside the chamber 30, while the second region 402 is located outside of the chamber 30. Thus, the user is allowed to pour water into the second region 402 directly when the water box 4 is not drawn out of the chamber 30, while to pour essential oil or to pour essential oil and water into the first region 401 when only a part of the water box 4 is drawn out of the chamber 30 to expose the first region 401 to the exterior of the chamber 30. In one embodiment, the water box 4 is provided with a water box cover 43. The water box cover 43 is provided with two water baffles 431, extending toward one of the water delivery pipelines 403, respectively. Each of the two water baffles 431 may be used for blocking one of the water delivery pipelines 403 incompletely, respectively, such that essential oil floating above water in the first region 401 may be blocked to be prevented from flowing toward the second region 402 by the two water baffles 431. In another embodiment, the water box 4 is provided with a bottle cap adaptor 44. The bottle cap adaptor 44 may be mounted to the top of the second region 402, such that a water bottle 6 may be insertedly connected to the bottle cap adaptor 44, so as to make up water into the water box 4 continuously. Furthermore, the bottle cap adaptor 44 may be also fitted with a plug when the bottle cap adaptor 44 is not insertedly connected to the water bottle 6, such that the bottle cap adaptor 44 may be used, as the water box cover 43, for preventing external dirt from dropping into the second region 402.

It is obviously found in the above paragraphs that the water box 4 may be placed into the chamber 30 through the gap 50 at the side of the decorative cover 5 without lifting the whole sweet atmosphere humidifier through placing the control module 2 and the air-guiding cap 3 on the surface of the base 1, and fitting all of the base 1, the control module 2 and the air-guiding cap 3 to the decorative cover 5. In this sweet atmosphere humidifier, moreover, the atomizing unit 41 is possibly provided in the water box 4 to facilitate replacement of the damaged atomizing unit 41 through fitting the first conductive terminal 33 to the second conductive terminal 42. Finally, the appearance profile of the decorative cover 5 may be further manufactured to fit the combined shape of the base 1, the control module 2, the air-guiding cap 3 and the water box 4, such that the part of the decorative cover 5 covering the base 1, the control module 2, the air-guiding cap 3 and the water box 4 may be formed as an appearance profile, like a mountain, ship, car, human body and etc., while the exhaust vent 32 of the air-guiding cap 3 is aligned with a crater, mouth, gun muzzle or any hole with meaningful appearance (for example, the through-hole 521) formed on the decorative cover 5, so as to obtain more interesting appearance of the present invention.

What is claimed is:

1. A sweet atmosphere humidifier, comprising:
a base;
a control module, provided on said base;
an air-guiding cap, provided on said base, said air-guiding cap being provided with a chamber formed within said air-guiding cap, an installation mouth communicated with said chamber, an exhaust vent communicated with said chamber and provided on the top of said air-guiding cap, and a first conductive terminal exposed inside said chamber and electrically connected to said control module;
a water box, selectively placed into said chamber through said installation mouth, said water box being provided with a liquid storage tank, an atomizing unit provided inside said liquid storage tank, and a second conductive terminal provided on the external surface of said water box while contacted with said first conductive terminal after said water box is placed into said chamber such that electric power is obtained by said atomizing unit; and
a decorative cover, mounted to said base so as to cover said control module, said air-guiding cap and said water box, said decorative cover being openly provided at the side thereof with a gap allowing said water box to pass there through and to be placed into or drawn out of said chamber.

2. The sweet atmosphere humidifier according to claim 1, wherein said water box is provided with a water box cover mounted to a part of said liquid storage tank exposed outside of said chamber.

3. The sweet atmosphere humidifier according to claim 1, wherein said water box is provided with a bottle cap adaptor, which is mounted to a part of said liquid storage tank exposed outside of said chamber and is provide for a water bottle to be mounted thereto.

4. The sweet atmosphere humidifier according to claim 1, wherein said liquid storage tank is provided with a first region located inside said chamber, a second region located outside of said chamber, and at least one water delivery pipeline located between said first region and said second region.

5. The sweet atmosphere humidifier according to claim 1, wherein said control module is provided with a switch element, which is electrically connected to said first conductive terminal and is capable of controlling said atomizing unit via said first conductive terminal and said second conductive terminal.

6. The sweet atmosphere humidifier according to claim 5, wherein said control module is provided with a power connecting port, which is electrically connected to said first conductive terminal and is provided for a power supply device to be electrically connected thereto so as to obtain electric power.

7. The sweet atmosphere humidifier according to claim 1, wherein said base and said air-guiding cap are integrally formed.

8. The sweet atmosphere humidifier according to claim 1, wherein said decorative cover is provided with a side cover, which is mounted to said gap and provided for covering one side of said water box.

9. The sweet atmosphere humidifier according to claim 1, wherein said chamber is provided with an installation channel communicated with said installation mouth and provided for said water box to be placed therein, and an air-guiding space communicated with said installation channel and provided for said exhaust vent to be located at the center of the top thereof.
